(19) United States
(12) Patent Application Publication
(10) Pub. No.: US 2006/0198849 A1
(43) Pub. Date: Sep. 7, 2006

(54) Preparation method of igy for preventing and cure mouth disease and the toothpaste base on the igy

(76) Inventors: Shing Paau, Admiraity (HK); Rong-Jiang Yang, Guangdong (CN)

Correspondence Address: Raymond Y Chan, Suite 128, 108 North Ynez Avenue, Monterey Park, CA, 91754, US

(21) Appl. No.: 10/547,237

(22) PCT Filed: Feb. 25, 2003
(86) PCT No.: PCT/CN03/00149

§ 371 Date: Aug. 25, 2005

(51) Int. Cl. A61K 39/40; A61K 39/116; C12P 21/04; A61K 8/96

(52) U.S. Cl. 424/164.1; 424/203.1; 435/70.21; 424/50

(57) ABSTRACT

The present invention relates to a preparation method of IgY for preventing and cure mouth disease, and a safe toothpaste containing the IgY.

### BACKGROUND OF THE PRESENT INVENTION

1. Field of Invention

The present invention relates to the preparation of IgY (egg yolk immunoglobulin) for the prevention and treatment of oral diseases, wherein further relates to the production of immuno-safe-toothpaste against oral diseases by using the IgY as a major ingredient

2. Description of Related Arts

Recently, there are various kinds of toothpaste, but they usually contain chemicals that is harmful to human beings, such as Sodium Dodecyl Sulfonate (SDS), Sodium Dodecyl Sarcosinate, Dodecyl Ethylene amide, Polyacrylic acid with Sodium polyacrylate, etc. Therefore, ingestion or swallowing of such toothpastes should be prevented while brushing teeth. Unfortunately, research had shown that adults could accidentally ingest 15-20% of the toothpaste used in terms of percentage volume while they are brushing teeth; a result of 40% had be shown for children in the same research. The pro-long use of such toothpaste is chronologically harmful to the body, and which is more serious to children and teenagers. The influence should not be neglected.

Furthermore, toothpastes recently on the market that are relatively effective contains fluoride, this chemical treatment cannot overcome the problem from the root and raised other problems:

(1) the occurrence mottled enamel: fluoride was added to tape water in Hong Kong and Guangzhou in 1961 and 1965 respectively to prevent dental caries, as a result, mottled enamel bloom;

(2) a research was carried out with observing children using fluorinated toothpaste for two months, results indicated that over-fluoride-absorption occurred in the children as the amount of toothpaste used varies and swallowing of toothpaste is serious. It is harmful to the health of children, especially those living in high-fluorine area;

(3) the pro-long use of fluoride will bring the dental caries Streptococcus to mutate, fluorine-resistant mutant strain can tolerate high fluorine concentration and even having higher caries ability, thus, worsen caries.

Moreover, most of the toothpastes designed for periodontitis and halitosis recently in the market contains wide-spectrum chemical germicides. These chemical germicides kill probiotic bacteria together with the pathogens in the oral cavity, which is seriously altering the oral micro-ecosystem. The effective period of these germicides only last for a few hours after teeth brushing, and pathogens bloom again after the hours. In addition, the pro-long use of chemical germicide leads pathogens to build tolerance, which finally worsen the fact.

To prepare IgY, organic solvents such as octanoic acid, alcohol, acetone, chloroform should be used during the extraction process in the classical methods recently. These solvents affect the activity of IgY and chemical residues generated are harmful to health. Besides organic solvents, precipitation is another preparation method. Polyethylene glycol, Dextran sulfate are common precipitation inducers, but the yield from is low and there is chemical residue also. The most recent method is water dilution extraction. The advantage of this method is having simple processes, high yield, low cost, no chemical hazards and safe, on the other hand, this method cannot remove excessive fat.

### SUMMARY OF THE PRESENT INVENTION

Targeted on the inadequate of the recent technologies, this invention is to provide a new IgY preparation method, and make this IgY to be an ingredient of a toothpaste that could control and treat oral diseases.

To achieve the recited objective, this invention include the methods to prepare IgY to control and treat oral diseases, wherein comprises:

(1) the incubation of pathogenic bacteria: using conventional practice to incubate major pathogenic bacteria regarding to dental caries, periodontitis and halitosis;

(2) the production of antigen complex: produce antigen complex from the pathogenic bacteria incubated;

(3) the injection: inject the antigen complex to egg-laying avian (e.g. hens, ducks, turkeys, etc.);

(4) the obtainment of immunized egg: analyze and pick immunized eggs laid by immunized avian;

(5) the production of egg yolk immunoglobulin: prepare egg yolk immunoglobulin from the egg yolk of the immunized eggs collected.

The method (1) of this invention, type C, D and G of Streptococcus mutans are incubated and obtained based on the pathogens arising dental caries; Fusobacterium nucleatum, Porphyromonas gingivalis, Actinomyces viscosus, Actinobacillus actinomycetemcomitans, Capnocytophaga ochracea, Treponemas denricola and Bacteroides forsythus are incubated and obtained based on the pathogens arising periodontitis and halitosis.

The method (5) in this invention, the preparation of IgY comprises:

(1) the extraction of egg yolk from the immunized eggs using “egg yolk sieve”, and mixing the extraction thoroughly;

(2) the addition of distilled water 4-6 times to the volume of the extraction;

(3) the adjustment of the pH of the mixture within 4.5-6.5;

(4) the addition of 2% Sodium alginate and make its concentration in the mixture within 1.0%-2.0%, and stir to precipitation;

(5) the addition of 2% CaCl₂ and make its concentration in the mixture within 0.5%-1.0%, and stirring thoroughly;

(6) the positioning of the mixture in 2-6° C. for 8-12 hours, an the siphoning of the supernatant;

(7) the centrifugation of the supernatant in (6) for 20 minutes at 8,000-12,000 rpm, and the taking of the supernatant;

(8) the ultra filtration for the supernatant in (7), and the sterilization with 0.22 μmembrane filter; and

(9) the freeze drying and the obtainment of the IgY extract.

The product in this invention, using the recited IgY to produce toothpaste, wherein contains 0.01-10.0% of the recited immunoglobulin, and:

(1) 0.3-5.0% foaming agent,

(2) 0.2-2.0% Sodium alginate,

(3) 0.5-5.0% Carboxymethylcellulose sodium,

(4) 0.12-20.0% Glycerol,

(5) 1.0-20.0% Sorbitol,

(6) 0.12-0.5% Aspartame, and

(7) appropriate ratio of flavorings.

The method (5) in this invention, the preparation of IgY comprises the followings.

In order to minimize the altering of foaming agent to the activity of IgY, foaming agent that does not denature protein could be used, the foaming agent could be Hydrogenated Castor Oil Polyoxyethylene Ether, Sodium N-lauroylamide ethanoate, or sodium N-lauroyl sarcosinate or the combination of either of the recited foaming agent. Appropriate ratio of oral mucosa adsorbent could also be added, such as 0.5-5.0% Caborpl.

To enhance the life of IgY within the paste, the IgY could be coated by water insoluble inert membrane, which does not affect the activity of the immunoglobulin, as micro-capsule, and make IgY distributed thoroughly throughout the paste.

The IgY prepared with the methods in this invention has high activity, high specificity, high effectiveness and safe, the processes are simple, low-cost and environmental friendly, and furthermore, the integrated use of side products such as egg shells, egg white and the other parts of egg yolk is another advantage. The brand new safe-toothpaste using the IgY as the major ingredient is having effective result on the control and treatment of caries, periodontitis and halitosis, and accidental ingestion is safe as there is no harmful chemicals within the toothpaste.

These and other objectives, features, and advantages of the present invention will become apparent from the following detailed description, the accompanying drawings, and the appended claims.

### DETAILED DESCRIPTION OF THE PREFERRED EMBODIMENT

1. The Preparation Procedures of IgY Complex:

This invention is applied with the principle of passive-immunity, IgY against specific pathogens could be obtained from the egg laid by egg-laying avian that immunized with appropriate amount of the specific antigen. The content of this sample comprises the incubation of the major pathogens with conventional practice with reference to the pathogens arising caries, periodontitis and halitosis, which is to make the IgY prepare is specific on the recited pathogens.

For the pathogens arising caries, serotype C, D and G of Streptococcus mutans could be selected. Mix the incubated S.mutans of different serotypes and add Freund adjuvant at the ratio 1:1; after the shattering and mixing of the mixture with a liquid mixer up to 10,000-30,000 rpm, antigen complex of S.mutans is prepared.

Besides caries, the following bacteria arising periodontitis and halitosis could be selected:

(1) Actinobacillus actinomycetemcomitans,

(2) Fusobacterium nucleatum,

(3) Porphyromonas gingivalis,

(4) Actinomyces viscosus,

(5) Capnocytophaga ochracea,

(6) Treponemas denricola, and

(7) Bacteroides forsythus, etc.

Mix the 7 representative pathogens that arising periodontitis and halitosis in appropriate ratio and add Freund adjuvant at the ratio 1:1; after the shattering and mixing of the mixture with a liquid mixer up to 10,000-30,000 rpm, antigen complex with whole bacterium and bacterial contents of multiple pathogens arising periodontitis and halitosis is prepared. Antigen complex arising halitosis only could also be prepared with several bacteria related to halitosis such as Fusobacterium nucleatum, Porphyromonas gingivalis, and Actinomyces viscosus by using the same recited procedures.

Perform hypodermic injections or wings-intravenous injections with one or several antigen complexes prepared from the above methods separately into well-breed and egg-laying hens, ducks or turkey that is having high sensitivity immune respond and selected by experiments. Second injection should be performed two weeks after the first injection, and three injections per avian in total. The eggs lay by the hens, ducks or turkeys 20 days after the first injection could be collected, the immunized eggs should be labeled differently according to the antigen injected.

The immunized eggs will be treated with the following processes:

(1) Extract the egg yolk from the immunized eggs using “egg yolk sieve”, and mix the extraction thoroughly;

(2) add distilled water 4-6 times to the volume of the extraction;

(3) adjust the pH of the mixture within 4.5-6.5;

(4) add 2% Sodium alginate (Food graded) and make its concentration in the mixture within 1.0%-2.0%, and stir to precipitation;

(5) add 2% CaCl2 (Food Graded) and make its concentration in the mixture within 0.5%-1.0%, and stir thoroughly;

(6) place the mixture in 2-6° C. for 8-12 hours, siphon the supernatant;

(7) centrifuge the supernatant in (6) for 20 minutes at 8,000-12,000 rpm, and take the supernatant;

(8) perform ultra filtration for the supernatant in (7), and sterile with 0.22 μmembrane filter; and

(9) freeze dry and obtain the IgY extract.

2. Experimental Researches Regarding the IgY Prepared Above

(1) Chronic toxicity test: Feed selected, health Sprague-Dawlye (SD) rats with the above IgY with 80 mg IgY/kg body weight/day for 30 days. Results had shown that rats did not carry any clinical symptoms, blood and urine tests resulted normal, liver and kidney test resulted normal, pathological surgery and tests on brain, heart, liver, kidney and testes did not find any abnormality.

(2) Activity test: Valence of Indirect Hemagglutination Inhibition Test was 1:512, and which of Enzyme-linked Immunosorbent Assay was 204,800 units.

(3) Adhesion inhibition test: Even the dilution factor of IgY reaches 1:8, the IgY still perform effective adhesion and inhibition to oral pathogens such as S.mutans, Actinomyces viscosus and Actinobacillus actinomycetemcomitans.

(4) Animal caries-prevention test: The above IgY was given to 10 rats in experimental group and placebo was given to 10 rats in control group. Results indicate that using the IgY of this invention to treat S.mutans infected rats could prevent the rats from dental caries. See table 1.

TABLE 1

| Test | Control | Experimental |
|---|---|---|
| Caries on Enamel only | 47 | 19 |
| Caries deep to ¼ of dentin | 25 | 0.57 |
| Caries deep to the whole dentin | 8 | 0 |

By, statistical analysis, P<0.01, difference between two groups is significant.

From results of the above experiments, the IgY prepared using methods from this invention is having high activity, high specificity, high effectiveness and safe, the processes are simple, low-cost and environmental friendly, and furthermore, the integrated use of side products such as egg shells, egg white and the other parts of egg yolk is another advantage.

3. IgY Safe-Toothpaste

The product, IgY Safe-toothpaste, of this invention is a brand new toothpaste that invented by targeting on the inadequateness of the classic toothpastes (most of the toothpastes recently in the market), which the IgY safe-toothpaste:

(1) is having IgY, which prepared with targeting on pathogens, as its major ingredient, which is harmless and do not come with toxic or side effects;

(2) is an edible toothpaste that has no harmful chemical, and having completely different ingredient with the classic toothpaste;

(3) is significantly effective as it is specific on the pathogens;

(4) is safe, edible, mouth-rinsing is not required after brushing teeth, thus, its convenient from in-house to travel and especially suitable for children.

Toothpastes now in the market contain various chemicals, especially foaming agents, such as Sodium Lauryl Sulfate, Sodium Dodecyl Sarcosinate, Lauroyl Diethanolamide, and Sodium Polyacrylate are strongly destructive to the activity of IgY. Result of an experiment that adding IgY to toothpaste without foaming agent in the concentration of 0.15% had shown that the IgY activity in the toothpaste can keep for a very long period of time. But, the activity of IgY had dropped from 1:64 to 1:8 in 1 minute, and decreased to 1:2 in 3 minutes, and finally approach zero in 10 minutes in the same experiment with the toothpaste replaced to classic toothpaste that contains foaming agent. It is significant that if the destruction effect on the activity of IgY from the chemicals, especially foaming agents, in toothpastes cannot be eliminated, IgY toothpaste will becomes valueless and meaningless.

With plenty of experiments, this invention uses a series of methods and measures to overcome the problems, makes the activity of IgY within the toothpaste become stable and lasting, and prepared an IgY safe-toothpaste which is highly active, highly stable and highly effective.

(1) Adjusting the Structure of the Toothpaste Tube

Make the classic, single-lumen tube a sandwich tube. This sandwich tube is composed with a set of two plastic tubes in different size and designated to fill with pastes with different contents. For example, the inner tube filled with the paste with IgY but do not come with foaming agent, and which the paste in the outer lumen contains foaming agent without IgY; and vise versa. When pressure is applied to the tube, paste in the inner lumen will be squeezed out from the center of the tube-neck, and the paste in the outer lumen will be squeezed out from the surrounding of the center of the tube-neck, making the pastes to combine. The design of the tube-neck could also be changed, for example, divide the circle exit into two semicircle, paste from the inner lumen will be coming out through one of the semicircle, and paste from the outer lumen through the other.

Besides the above designs, the tube could also being designed by using a thin layer plastic membrane as a partition to separate the paste with foaming agent (without IgY) and which with IgY (without foaming agent).

The designs above could separate IgY from the foaming agent until the pastes are squeezed out, thus, the activity of IgY inside the paste could be maintained.

(2) Using Mild Foaming Agent

Choose materials with foaming ability, and add into toothpaste with different ratio to test if the foaming effect reaches the standard requirement of toothpaste. Afterwards, the passed foaming materials will then mixed with IgY in different ratio, the best foaming material that most harmless to IgY was chosen to be the foaming agent based on the activity recorded at 1 min, 3 mins, 10 mins and 24 hrs.

After hundreds of experiments and tests, the best foaming agents are:

(i) Polyoxyethylene ether hydrogenated castor oil,

(ii) Sodium N-lauroylamide ethanoate, and

(iii) Sodium N-lauroyl sarcosinate

These foaming agents are having the least destructive effect on the activity of IgY and can generate sufficient foam when used in toothpaste. In tests that mixing the foaming agent with IgY and toothpaste, after 10 mins, 30 mins, 24 hrs, 30 days and 60 days, all of the above 3 foaming agents can keep the activity of IgY above 1:16 throughout the tests.

Another test was performed by placing the toothpastes (which the same as the previous test) in the temperature of 40° C., activity of the toothpaste was checked on day 15, 30, and 60, activity do only decrease insignificantly and keep high activity (≧1:16). All of the 3 selected foaming agents are mild, edible and none of toxic or side effects, accidental ingestion is harmless while brushing teeth with the toothpaste composed with either one of these foaming agents.

Experimental results shown that the selected foaming agents can fulfill the requirement of preparing IgY toothpaste.

(3) Coat with Micro-Capsule

To further stabilize the activity of IgY complex, a special micro-capsule coating technique, which comprise the coating of IgY by using a water insoluble inert material that do not affect the activity of IgY, could also be applied. This invention had discovered that, using a complex of Polybenzylamide and Polyethylene amide as the coating agent could do, or other materials that is inert, water insoluble and do not denature protein could also do. The coating agent selected is edible and haven't any toxic or side effects. The function of this coating agent is to wrap IgY with a thin layer as a capsule, make the IgY become suspended in the toothpaste and insulated to the reactions between IgY and the other chemicals in the toothpaste. When paste is squeezed out, the micro-capsule will break easily with the action of teeth brushing, thus IgY releases and functions.

One of the advantages of using the thin layer capsule to wrap IgY is further rising the stability of IgY in the toothpaste and thus increase the shelf-life; another advantage is the thin layer is easy to break, which means mild friction or pressure can release the wrapped IgY. These advantages give this brand new toothpaste practical effectiveness. On the other hand, if classic micro-capsule technique is used, IgY could also be protected, but its difficult to break and not all the IgY is released during teeth brush, resulting negative effects.

(4) Addition of Oral Mucosa Adsorbent

In this invention, oral mucosa adsorbent (slow-release formulation), such as Caborpl, is added to toothpaste for prolonging the time of pathogen inhibition and the effectiveness by keeping the IgY to stay on the oral mucosa longer.

(5) The Formulation of IgY Safe-Toothpaste

The following ingredients are measured in (w/w):

i. IgY preparation: 0.01-10.0%;

ii. Caborpl: 0.5-1.0%;

iii. Sodium N-lauroylamide ethanoate: 0.3-5.0%;

iv. Sodium alginate: 0.2-2.0%;

v. Sodium carboxy methylcellulose: 0.5-5.0%;

vi. Glycerol: 0.12-20.0%;

vii. Sorbitol: 1.0-20.0%;

viii. Aspartame: 0.12-0.50%;

ix. Menthol essence: 0.1-0.5%;

x. Orange essence: 0.1-0.5%;

xi. Peach essence: 0.1-0.5%;

xii. Vanilla essence: 0.1-0.5%.

All the ingredients must be food-graded. Food preservative could be added according to the designated shelf-life. This formula was tested to be harmless to the activity of IgY.

In the following preferred embodiment, ingredients could be: (in (w/w))

i. IgY preparation: 5.0%;

ii. Caborpl: 0.5%;

iii. Sodium N-lauroylamide ethanoate: 2.5%;

iv. Sodium alginate: 3.0%;

v. Sodium carboxy methylcellulose: 2.5%;

vi. Glycerol: 10.0%;

vii. Sorbitol: 5.0%;

viii. Aspartame: 0.20%;

ix. Menthol essence: 0.25%;

x. Orange essence: 0.15%;

xi. Peach essence: 0.15%;

xii. Vanilla essence: 0.25%.

(6) Production Arts:

Melt iv and v in 60 mL of distilled water; add other ingredients one by one and mix thoroughly; add IgY as the final ingredient and mix; add distilled water to 100 mL and mix to cool paste formation; infuse the paste into tubes.

4. The Effectiveness Test of IgY Safe-Toothpaste

(1) Effectiveness Observation on Dental Caries Prevention:

To ensure the practical effectiveness of the above IgY Safe-toothpaste on caries prevention, clinical tests was done on population by the inventor. The mouth (oral cavity) of 60 non-caries volunteers were cleaned and volunteers are randomly divided into two groups with 30 volunteers each, which all the volunteers brush their teeth twice a day (in the morning and prior to bed) for 3 minutes each time, the experimental group uses the IgY toothpaste of this invention, and the volunteers of control group brush their teeth with classic toothpaste. Bacterial samples were picked from plaque and tongue surface and inoculated on TS medium (as the total anaerobic bacteria medium) and TSB medium (as a selective medium for S.mutans), CFU and the percentage S.mutans in total anaerobic bacteria was calculated after 36 hrs anaerobic incubation. Samples were taken once a week for 9 weeks, and the results are indicated in table 2.

Percentage S. mutans in total anaerobic bacteria

| | Experimental Group | | Control Group | |
|---|---|---|---|---|
| Week | Saliva (%) | Plaque (%) | Saliva (%) | Plaque (%) |
| 0 | 56.23 | 56.43 | 52.56 | 50.10 |
| 1 | 45.60 | 42.28 | 49.10 | 48.10 |
| 2 | 41.80 | 38.39 | 49.20 | 47.08 |
| 3 | 32.83 | 30.15 | 48.10 | 46.15 |
| 4 | 28.15 | 19.10 | 47.70 | 43.60 |
| 5 | 25.29 | 18.32 | 43.19 | 45.55 |
| 6 | 23.19 | 17.92 | 49.20 | 46.35 |
| 7 | 21.08 | 15.10 | 50.05 | 50.26 |
| 8 | 20.05 | 13.96 | 51.16 | 49.18 |

For weeks after the starting day, the experimental group uses the IgY toothpaste in this invention got a deep drop of percentage S. mutans in total anaerobic bacteria for both saliva and plaque; with statistical analysis, result=P<0.01, reflect that there is significant change before and after experiment. On the other hand, the control group which uses classic toothpaste remains unchanged, statistical analysis results P>0.05, reflect no significant change. The results indicate that the IgY toothpaste of this invention can significantly inhibit S. mutans in saliva and plaque, thus having specific effectiveness on preventing caries.

(2) Specific Inhibition Effectiveness Observation on Pathogen Arising Periodontitis:

100 periodontitis mid-age volunteers with mid-phase periodontitis and similar symptoms were selected and the mouth (oral cavity) of all volunteers are cleaned. Volunteers are randomly divided into two groups of 50 volunteers, which all the volunteers brush their teeth conscientiously twice a day (in the morning and prior to bed) for 3 minutes each time, the experimental group uses the IgY toothpaste of this invention, and the volunteers of control group brush their teeth with classic toothpaste. Bacterial samples were picked from periodontal area, and CFU was calculated after 36 hrs anaerobic incubation. CFU of week 0 was set to be 100%, the result of every sampling was compare with which of week 0 (relative CFU), and tabulated below:

TABLE 3
Relative CFU variation of major periodontitis pathogens (%)

| | Experimental Group | Control Group |
|---|---|---|
| Week | Relative CFU | Relative CFU |
| 0 | 1 | 1 |
| 1 | 0.90 | 0.98 |
| 2 | 0.70 | 0.90 |
| 3 | 0.65 | 0.92 |
| 4 | 0.60 | 0.86 |
| 5 | 0.52 | 0.88 |
| 6 | 0.50 | 0.85 |
| 7 | 0.45 | 0.86 |
| 8 | 0.38 | 0.90 |

Relative CFU is the comparative result between CFU of sample taken at every week and which of week 0 (illustrated in percentage).

From the analysis above, major pathogens arising periodontitis found in the periodontal area dropped deeply in experimental group, which uses the IgY toothpaste of this invention, after 4 weeks, it drop from 100% to 60%, and further decreased to 38% at week 8 in terms of relative CFU. With statistical analysis, P<0.01, represent significant difference. On the other hand, the relative CFU of control group, which uses classic toothpaste, remains unchanged, statistical analysis reflect no significant change as P>0.05. The results indicate that the IgY toothpaste of this invention can perform specific inhibition on the pathogens causing periodontitis significantly.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method of producing an egg yolk immunoglobulin (IgY) for the control and treatment of oral diseases, comprising the steps of:
(1) incubating a pathogenic bacteria by using a conventional practice to incubate major pathogenic bacteria regarding to dental caries, periodontitis and halitosis;
(2) producing an antigen complex by producing antigen complex from said pathogenic bacteria incubated in step (1);
(3) carrying out an injection by injecting said antigen complex to an egg-laying avian (such as hens, ducks, turkeys, etc.);
(4) obtaining an immunized egg by analyzing and picking immunized eggs laid by said immunized avian; and
(5) producing an egg yolk immunoglobulin by preparing said egg yolk immunoglobulin from egg yolk of said immunized eggs collected.

2. The method, as recited in claim 1, wherein in step (1) further comprises a step of selecting a procedure to incubate and obtain type C, D and G of Streptococcus mutans based on the pathogens arising dental caries.

3. The method, as recited in claim 1, wherein in step (1) further comprises a step of selecting a procedure to incubate and obtain Fusobacterium nucleatum, Porphyromonas gingivalis and Actinomyces viscosus based on the pathogens arising periodontitis and halitosis.

4. The method, as recited in claim 3, wherein in step (1) further comprises a step of selecting a procedure to incubate and obtain Actinobacillus actinomycetemcomitans, Capnocytophaga ochracea, Treponemas denricola and Bacteroides forsythus based on the pathogens arising periodontitis and halitosis.

5. The method, as recited in claims 1, 2, 3 or 4, wherein in step (2) further comprises the steps of:
(2.1) mixing of said incubated bacteria with a specific ratio;
(2.2) adding of Freund adjuvant with the ratio 1:1;
(2.3) shattering and mixing of the mixture with a liquid mixer up to 10,000-30,000 rpm; and
(2.4) producing an antigen complex related to dental caries, periodontitis and halitosis separately and respectively.

6. The method, as recited in claims 1, 2, 3 or 4, wherein in step (5) comprises the steps of:
(5.1) extracting an egg yolk from said immunized eggs using “egg yolk sieve”, and mixing the extraction thoroughly;
(5.2) adding distilled water 4-6 times to the volume of the extraction;
(5.3) adjusting the pH of the mixture within 4.5-6.5;
(5.4) adding 2% Sodium alginate and making its concentration in the mixture within 1.0%-2.0%, and stirring to precipitation;
(5.5) adding 2% CaCl₂ and making its concentration in the mixture within 0.5%-1.0%, and stirring thoroughly;
(5.6) positioning the mixture in 2-6° C. for 8-12 hours and siphoning the supernatant;
(5.7) conducting the centrifugation of the supernatant in step (6) for 20 minutes at 8,000-12,000 rpm and taking the supernatant;
(5.8) conducting ultra filtration for the supernatant in step (7), and conducting sterilization with 0.22 μm membrane filter; and
(5.9) freeze drying and obtaining an IgY extract.

7. A toothpaste using said immunoglobulin (IgY) produced in claim 1 as one of its raw materials, comprising a paste containing 0.01-10.0% of said immunoglobulin.

8. The toothpaste, as recited in claim 7, wherein said immunoglobulin used can be produced by the method as recited in claims 2, 3 or 4, or by the method as recited in claims 1, 5 or 6, or by a combination of said immunoglobulin in claims 1-6.

9. The toothpaste, as recited in claim 7, wherein said paste comprises the ingredients consisting of:
(1) 0.3-5.0% foaming agent;
(2) 0.2-2.0% Sodium alginate;
(3) 0.5-5.0% carboxymethylcellulose sodium;
(4) 0.12-20.0% glycerol;
(5) 1.0-20.0% sorbitol;
(6) 0.12-0.5% aspartame; and
(7) an appropriate ratio of flavorings.

10. The toothpaste, as recited in claim 9, wherein said foaming agent does not denature protein and said foaming agent can be Hydrogenated Castor Oil Polyoxyethylene Ether, Sodium N-lauroylamide ethanoate or Sodium N-lauroyl sarcosinate, or a combination of said Hydrogenated Castor Oil Polyoxyethylene Ether, Sodium N-lauroylamide ethanoate or Sodium N-lauroyl sarcosinate.

11. The toothpaste, as recited in claim 10, further comprising an appropriate ratio of oral mucosa adsorbent, wherein said adsorbent can be 0.5-5.0% Caborpl.

12. The toothpaste, as recited in claim 11, wherein said flavorings consists of:
(1) 0.1-0.5% menthol essence;
(2) 0.1-0.5% orange essence;
(3) 0.1-0.5% peach essence; and
(4) 0.1-0.5% vanilla essence.

13. The toothpaste, as recited in claims 7, 8, 9, 10, 11 or 12, wherein said immunoglobulin is coated by water insoluble inert membrane which does not affect the activity of the immunoglobulin as micro-granulates, and is distributed thoroughly throughout said paste.

14. The toothpaste, as recited in claim 13, wherein said membrane can be a complex of polybenzylamide and polyethylene amide.

15. The toothpaste, as recited in claims 7, 8, 9, 10, 11 or 12, comprising a packing tube, wherein said packing tube comprises a sandwich layer having an inner-lumen and an outer-lumen, wherein said inner-lumen can be used to fill said paste with said immunoglobulin without foaming agent, said outer-lumen is filled with said paste that contain said foaming agent with no immunoglobulin, and vice versa.

16. The toothpaste, as recited in 7, 8, 9, 10, 11 or 12, comprising a packing tube, wherein said packing tube can be separated with a thin layer to form two partitions, that one of said partitions can be filled with said paste with said immunoglobulin containing no foaming agent, and that the other one partition is filled with said paste containing foaming agent without said immunoglobulin.
